(19) United States
(12) Patent Application Publication
(10) Pub. No.: US 2024/0315248 A1
(43) Pub. Date: Sep. 26, 2024

(54) NOVEL ANTIMICROBIAL COMPOSITIONS AND ARTICLES MADE THEREFROM

(71) Applicant: 3M INNOVATIVE PROPERTIES COMPANY, Saint Paul, MN (US)

(72) Inventors: Vinod P. MENON, Woodbury, MA (US); Bonnie J. HOVDE, Lake Elmo, MN (US); James P. DIZIO, St. Paul, MN (US); Korey KARLS, Woodbury, MN (US)

(21) Appl. No.: 18/259,555

(22) PCT Filed: Dec. 15, 2021
(86) PCT No.: PCT/IB2021/061784

§ 371 Date: Jun. 27, 2023

Related U.S. Application Data
(60) Provisional application No. 63/131,876, filed on Dec. 30, 2020.

(51) Int. Cl. A01N 47/44; A01N 25/10; A01N 25/24; A01N 43/40; A61L 24/00; A61L 24/04

(52) U.S. Cl. CPC A01N 47/44; A01N 25/10; A01N 25/24; A01N 43/40; A61L 24/0021; A61L 24/046; A61L 2300/404

(57) ABSTRACT

Antimicrobial compositions and articles are disclosed, as well as method for preparing antimicrobial compositions. Also disclosed are method for using antimicrobial compositions and articles for preventing the formation or growth of biofilms, disrupting biofilms, reducing microorganism counts, and treating infections.

### BACKGROUND

Despite advances made in infection-control practices, surgical-site infections (SSIs) remain a substantial cause of morbidity, prolonged hospitalization, and death. In fact, SSIs are associated with a mortality rate of 3% and 75% of SSI-related deaths are directly attributable to the SSI. Surgeons currently rely on surgical drapes having iodine-impregnated adhesives to mitigate contact with pathogenic microbes. While povidone-iodine is a widely effective antiseptic, there are drawbacks to its use. For example, povidone-iodine may cause skin irritations in some individuals, and use with large wounds may lead to kidney problems, high blood sodium, and metabolic acidosis. Furthermore, the use of povidone-iodine is not recommended for those that are less than 32 weeks pregnant, those that are prescribed lithium, or those with thyroid problems. Chlorhexidine gluconate and octendine hydrochloride are a viable alternative that is not associated with the aforementioned fallbacks.

Developing antiseptic-impregnated adhesives (e.g., chlorhexidine gluconate- and octenidine hydrochloride-impregnated adhesives) would help reduce surgical-site infection rates, meanwhile potentially avoiding the side effects associated with povidone-iodine. What is needed are pressure-sensitive adhesives capable of carrying and delivering chlorhexidine gluconate, octenidine hydrochloride, or a combination thereof.

### SUMMARY

In one embodiment, a composition is described. The composition includes chlorhexidine gluconate, octenidine hydrochloride, or a combination thereof and a hydrophobic plasticizer capable of solubilizing chlorhexidine gluconate, octenidine hydrochloride, or a combination thereof in an amount at least about 2 g chlorhexidine gluconate, octenidine hydrochloride, or a combination thereof per 100 g hydrophobic plasticizer at a temperature of about 20-23° C. The hydrophobic plasticizer has at least two hydrogen-bonding groups separated by greater than three consecutive atoms and is characterized by an HLB of no greater than 10 as determined using the HLB Method.

In one embodiment, an antimicrobial adhesive is described. The antimicrobial adhesive includes any composition described herein and a pressure-sensitive adhesive.

In one embodiment, a medical article is described. The medical article includes a substrate and any antimicrobial adhesive described herein disposed on the substrate.

In one embodiment, a method for preparing a composition described herein is described. The method includes providing chlorhexidine gluconate, octenidine hydrochloride, or a combination thereof and the hydrophobic plasticizer, and contacting the chlorhexidine gluconate, octenidine hydrochloride, or a combination thereof to the hydrophobic plasticizer to form the composition.

In one embodiment, a method for preparing an antimicrobial adhesive described herein is described. The method includes providing a composition described herein and a pressure-sensitive adhesive, and contacting the composition and the pressure-sensitive adhesive to form the antimicrobial adhesive.

In one embodiment, a method for preparing a medical article described herein is described. The method includes providing an antimicrobial adhesive described herein and a substrate, and coating the antimicrobial adhesive onto the substrate.

In one embodiment, a method for disinfecting a surface is described. The method includes providing a medical article described herein, and contacting the medical article to the surface for a period.

In one embodiment, a method for preparing a surface for surgery is described. The method may include providing a medical article described herein, and contacting the medical article to the surface.

In one embodiment, a kit is described. The kit includes a medical article described herein and a set of instructions directing a user to disinfect a surface according to the methods described herein.

### DETAILED DESCRIPTION

Efforts to develop antiseptic-impregnated adhesives have been met with significant challenges. Chlorhexidine gluconate (“CHG”) and octenidine hydrochloride (“octenidine”), each being a highly polar compounds, tend to precipitate from hydrophobic adhesive compositions. The lack of CHG solubility or octenidine solubility in the adhesive effectively immobilizes CHG or octenidine such that it is unavailable for adequate transfer to a surface. Moreover, blending additives like CHG and CHG-solubilizing vehicles often compromise the strength of the adhesive, which leads to premature adhesive failure. In terms of surgical drapes, this premature adhesive failure is called ‘drape drift.’ When surgical drapes move or ‘drift,’ the patient experiences a greater exposure to microbes and becomes more vulnerable to infection.

The present disclosure is directed toward chlorhexidine gluconate-based and octenidine hydrochloride-based compositions for homogenous inclusion within pressure-sensitive adhesive (PSA) formulations, and medical articles made therefrom. Initially, it was believed that hydrophilic (polar) vehicles were required to render chlorhexidine gluconate or octenidine hydrochloride compatible with adhesives. It was later found that hydrophobic (non-polar) vehicles having vicinal (i.e., separated by two atoms; adjacent), or otherwise proximate (i.e., separated by three atoms), hydrogen-bonding groups were effective at solubilizing chlorhexidine gluconate and octenidine hydrochloride, which in turn compatibilized the hydrophobic CHG solutions and hydrophobic octenidine solutions with hydrophobic pressure-sensitive adhesives (see WO2014/035981). In this work, it was surprisingly found that CHG and octenidine, despite being polar compounds, are readily soluble in hydrophobic plasticizers that have hydrogen-bonding groups spaced more than three atoms apart. In addition, it was surprisingly found that hydrophobic vehicles bearing vicinal hydrogen-bonding groups can be detrimental to adhesive integrity as compared to compositions that are void of said hydrophobic vehicles. The long-sought means to incorporate CHG and/or octenidine hydrochloride into PSA-containing medical articles, such as surgical drapes, is presented herein.

As used herein, “about” means ±10 percent of a given value. For example, about 10 means 9 to 11.

As used herein, “acid” refers to a carboxylic acid group, i.e., —CO₂H.

As used herein, “alkyl” refers to a straight or branched saturated hydrocarbon group. “C₁₋₆ alkyl” denotes the number of carbon atoms within the hydrocarbon group. For example, C₁₋₆ means 1-6 carbons, e.g., methyl, ethyl, propyl, isopropyl, butyl, isobutyl, sec-butyl, pentyl, hexyl, and the like.

As used herein, “alkyl diol” refers to a straight or branched saturated hydrocarbon group bearing two hydroxyl (—OH) groups. For example, a C₅ alkyl 1,2-diol means CH₃CH₂CH₂CH(OH)CH₂(OH), CH₃CH(CH₃)CH(OH)CH₂(OH), or the like. For example, a C₅ alkyl 1,3-diol means CH₃CH₂CH(OH)CH₂CH₂(OH), or the like. An “alkyl triol” refers to a straight or branched saturated hydrocarbon group bearing three hydroxyl (—OH) groups.

As used herein, “alkylene” refers to a straight or branched bivalent hydrocarbon group. For example C₆ alkylene means —CH₂CH₂CH₂CH₂CH₂CH₂—, —CH₂CH(CH₂CH₃)CH₂CH₂—, or the like.

As used herein, “alkenyl” refers to a straight or branched unsaturated hydrocarbon group. For example, a C₄ alkenyl means —CH₂═CHCH₂CH₃, —CH₂═C(CH₃)CH₃, —CH₂═CHCH₂═CH₂, or the like.

As used herein, “alkenylene” refers to a straight or branched bivalent unsaturated hydrocarbon group. For example, a C₄ alkenylene means —CH₂═CHCH₂CH₂—, —CH₂═C(CH₃)CH₂, —CH₂═CHCH₂═CH—, or the like.

As used herein, “consecutive atoms” refers to atoms, such as carbon, nitrogen, oxygen, and sulfur, that are linked together to form a chain. Groups that are separated by a number of consecutive atoms means that the chain includes that number of intervening atoms groups. For example, the hydroxyls in the following chains are separated by 4 consecutive atoms: HOCH₂CH₂CH(CH₃)CH₂OH, HOCH₂CH═CHCH₂OH, HOCH₂C(O)CH₂CH₂OH, or the like.

As used herein, “cyclohexylene” refers to a bivalent 6-membered cyclic hydrocarbon group.

As used herein, “cyclohexenylene” refers to a bivalent 6-membered cyclic hydrocarbon group having one or more units of unsaturation, i.e., —C(R)═C(R)—.

As used herein, “excluded” means that there is 0.0 wt % of the substance present in the composition.

As used herein, “derived” means that a component X may be prepared from compounds Y, e.g., a hydrophobic plasticizer (X) may be prepared (derived) from a C₁₆₋₄₄ alkyl acid compound (Y).

A skilled artisan would readily understand the synthetic methodologies suitable for reacting any described compounds Y herein. For example, a person of ordinary skill would understand that an organic acid (R^{a}CO₂H, R^{a} is arbitrary) and an alcohol (R^{b}—OH, R^{b} is arbitrary) may be combined to form esters (R^{a}CO₂R^{b}).

As used herein, “disinfecting” refers to a reduction in the number of active microorganisms present on a surface being disinfected. Disinfecting may kill or prevent microorganisms from growing or proliferating.

As used herein, “hydrogen-bonding group” refers to a group having an atom with lone pair of electrons and an electronegativity greater than carbon. An atom of such characterization may bond with a hydrogen atom bonded to another such group by a process known in the art as hydrogen bonding. For example, —OH, —NH₂ and —SH, —O—, —N(CH₃)— and —S— all are considering hydrogen-bonding groups.

As used herein, “hydrogen-bond donor” refers to a hydrogen-bonding group that includes a hydrogen atom bound to the atom with the lone pair of electrons. For example, —OH, —NH₂ and —SH are hydrogen-bond donors.

As used herein, “hydrophilic-lipophilic balance” or “HLB” values are calculated using the method of Griffin (Griffin WC; J. Soc. of Cosmetic Chemists 5, 259 (1954)). Thus, as used herein, the “HLB Method” involves a calculation based on the following:

$$HLB = 20 * {M}_{h} / M$$

where Mₕ is the molecular mass of the hydrophilic portion of the molecule, and M is the molecular mass of the whole molecule, giving a result on a scale of 0 to 20. An HLB value of 0 corresponds to a completely lipophilic/hydrophobic molecule, and a value of 20 corresponds to a completely hydrophilic/lipophobic molecule. As used in the present disclosure, hydrophilic vehicles have an HLB value as calculated using the HLB Method of greater than 10. In some embodiments, the hydrophilic vehicle has an HLB value of greater than 11, e.g., greater than 12. Hydrophobic vehicles have an HLB value as calculated using the HLB Method of no greater than 10. In some embodiments, the hydrophobic vehicle has an HLB value of no greater than 9, e.g., no greater than 7.

As used herein, “hydrophilic vehicle” refers to a compound characterized by an HLB value of greater than 10. A hydrophilic vehicle, as described by the present disclosure, is intended to include compounds having a molecular weight of no greater than 5 kg/mole. Furthermore, a hydrophilic vehicle, as described by the present disclosure, is not intended to include water.

As used herein, “hydrophobic vehicle” refers to a compound characterized by an HLB value of no greater than 10 and having two hydrogen-bonding groups (e.g., alkoxy separated by no more than three atoms, e.g., R—CH(X)CH₂(X), R—X—CH₂CH₂X, or the like.

As used herein, “hydroxyl value” refers to the measure of content of free hydroxyl groups in a chemical substance, expressed in units of mass of potassium hydroxide in milligrams equivalent to the hydroxyl content of one gram of the chemical substance.

As used herein, “optionally substituted” describes a chemical entity or group that may or may not be substituted with one or more recited chemical moieties. For example, a “C₁₋₆ alkyl group optionally substituted with one or more hydroxyl” is intended to include a non-substituted C₁₋₆ alkyl group or a C₁₋₆ alkyl group substituted with one or more —OH, e.g., 2-hydroxybutyl, or the like.

As used herein, “plasticizer” refers to a substance or combination of substances that lowers the glass transition temperature of another substance (e.g., a pressure-sensitive adhesive). Plasticizers effectively soften, increase flexibility, increase plasticity, decrease viscosity, and/or decrease friction of a substance to which it is added. The plasticizers described herein are relatively low molecular weight polymers that increase the spacing between chains of a larger molecular weight polymer (i.e., PSA).

As used herein, “polymer” refers to a substance having one or more repeating monomer units. The chemical identities of the polymeric substances herein are at times described in terms of the monomers to which the polymer is derived. A skilled artisan would readily understand the reactivity profile of the recited monomers and how the monomers could synthetically be joined to form the polymer.

As used herein, “pressure-sensitive adhesive” refers to a non-reactive, self-stick adhesive that forms a bond when pressure is applied. No solvent, water, or heat is required to activate a pressure-sensitive adhesive.

As used herein, “substituted” means replacement of any present C—H bond with the reported substitution group (X), i.e., C—H→C—X.

When referring to “solubility,” or “to solubilize” it should be understood that the solubility of a component A in a component B refers to conditions in which only component A and component B are present, e.g., no added salts, compounds, or the like. Furthermore, any solubility values provided herein are with regard to a temperature range of about 20° C. to about 23° C. at atmospheric pressure (i.e., 760 mm/Hg).

As used herein, “unsaturated” means that a substance includes a unit of unsaturation, i.e., two adjacent hydrogens are removed to form a pi bond between atoms. A unit of unsaturation includes alkenes (—C(R)═C(R)—) and alkynes (—C≡C—).

In shall be understood that any generalized chemical names shall include only the chemical groups to which are recited in the generalized chemical name, unless specifically stated otherwise, e.g., substituted, optionally substituted, or the like. For example, a C₁₈ alkyl acid consists of a Cis alkyl group and a carboxylic acid group. For example, a C₁₈ alkyl acid substituted with one or more hydroxyl consists of a C₁₈ alkyl group, a carboxylic acid group, and one or more hydroxyl substitution.

### Compositions

In various embodiments, a composition is described. The composition may include chlorhexidine gluconate and/or octenidine hydrochloride and a hydrophobic plasticizer capable of solubilizing chlorhexidine gluconate, octenidine hydrochloride, or a combination thereof in an amount at least about 2 g chlorhexidine gluconate, octenidine hydrochloride, or a combination thereof per 100 g hydrophobic plasticizer at a temperature of about 20-23° C. The hydrophobic plasticizer may include at least two hydrogen-bonding groups separated by greater than three consecutive atoms and may be characterized by an HLB of no greater than 10 as determined using the HLB Method.

In some embodiments, the composition may consist essentially of the chlorhexidine gluconate, octenidine hydrochloride, and the hydrophobic plasticizer. In some embodiments, the composition may consist essentially of the chlorhexidine gluconate and the hydrophobic plasticizer. In some embodiments, the composition may consist essentially of the octenidine hydrochloride and the hydrophobic plasticizer.

In some embodiments, the composition may include chlorhexidine gluconate, octenidine hydrochloride, or a combination thereof present in an amount of at least about 0.05 wt % with respect to the weight of the composition. In some embodiments, the composition may include chlorhexidine gluconate, octenidine hydrochloride, or a combination thereof present in an amount of no more than about 5 wt % with respect to the weight of the composition. The composition may include chlorhexidine gluconate, octenidine hydrochloride, or a combination thereof present in an amount (wt % with respect to the weight of the composition) of about 0.05, 0.1, 0.2, 0.3, 0.4, 0.5, 0.6, 0.7, 0.8, 0.9, 1.0, 1.2, 1.4, 1.6, 1.8, 2.0, 2.2, 2.4, 2.6, 2.8, 3.0, 3.2, 3.4, 3.6, 3.8, 4.0, 4.2, 4.4, 4.6, 4.8, 5.0, or a value within a range of any of the preceding values, for example, between about 0.2 and about 4.0, between about 2.0 and about 3.0, or the like.

In some embodiments, the composition may include chlorhexidine gluconate, octenidine hydrochloride, or a combination thereof present in an amount of at least about 0.1 wt % with respect to the weight of the hydrophobic plasticizer. In some embodiments, the composition may include chlorhexidine gluconate, octenidine hydrochloride, or a combination thereof present in an amount of no more than about 20 wt % with respect to the weight of the hydrophobic plasticizer. The composition may include chlorhexidine gluconate, octenidine hydrochloride, or a combination thereof present in an amount (wt % with respect to the weight of the hydrophobic plasticizer) of about 0.1, 0.5, 1, 2, 3, 4, 5, 6, 7, 8, 9, 10, 12, 14, 16, 18, or 20, or a value within a range of any of the preceding values, for example, between about 0.5 and about 12, between about 1 and about 10, or the like.

In some embodiments, the composition may include the hydrophobic plasticizer present in an amount of at least about 10 wt % with respect to the weight of the composition. In some embodiments, the composition may include the hydrophobic plasticizer present in an amount of no more than about 50 wt % with respect to the weight of the composition. The composition may include the hydrophobic plasticizer present in an amount (wt % with respect to the weight of the composition) of about 10, 15, 20, 25, 30, 35, 40, 45, or 50, or a value within a range of any of the preceding values, for example, between about 10 and about 25, between about 20 and about 40, or the like.

In some embodiments, the composition may further include water present in an amount of less than about 1 wt % with respect to the weight of the composition. In some embodiments, the composition may further include water (wt % with respect to the weight of the composition) present in an amount of about 0, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, or a value between any of the preceding values, for example, between about 0.01 and about 1, between about 0.1 and about 0.5, or the like.

In some embodiments, the composition may further include water present in an amount of less than about 1:1 by weight with respect to the weight of chlorhexidine gluconate, octenidine hydrochloride, or a combination thereof. In some embodiments, the composition may further include water present in an amount (with respect to the weight of chlorhexidine gluconate, octenidine hydrochloride, or a combination thereof) of less than about 1:1, less than about 0.9:1, less than about 0.8:1, less than about 0.7:1, less than about 0.6:1, less than about 0.5:1, less than about 0.4:1, less than about 0.3:1, less than about 0.2:1, less than about 0.1:1, less than about 0.05:1, less than about 0.1:1, 0:1, or a value between any of the preceding values, for example between about 0.5:1 and about 0.2:1, between about 0.9:1 and about 0.7:1, or the like.

In some embodiments, the composition may further include a hydrophilic vehicle characterized by an HLB of greater than 10 in an amount of less than about 0.1:1 with respect to the weight of chlorhexidine gluconate, octenidine hydrochloride, or a combination thereof. In some embodiments, the composition may include a hydrophilic vehicle in less than about 0.1:1, less than about 0.05:1, less than about 0.01:1, less than about 0.005:1, less than about 0.001:1, or a value between any of the preceding values. In some embodiments, the composition excludes (0:1 w/w) a hydrophilic vehicle.

In some embodiments, the composition may exclude C₂-C₂₂ alkyl 1,2-diols and C₃-C₂₂ alkyl 1,3-diols.

In some embodiments, the composition may exclude C₈₋₁₂ alkyl 1,2-diols and C₈₋₁₂ alkyl 1,3-diols.

In some embodiments, the composition may exclude compounds of the formula:

HO—C(R^{a})₂—C(R^{a})₂—OC(O)—R^{b},

wherein:
each R^{a} is independently —H or C₁₋₁₈ alkyl, and
R^{b} is C₄₋₂₂ alkyl.

In some embodiments, the composition may exclude compounds of the formulae:

HO—C(R^{c})₂—C(R^{c})(OH)—C(R^{c})₂—OC(O)—R^{c},

HO—C(R^{c})₂—C(R^{c})(OC(O)—R^{b})—C(R^{c})₂—OH, or

HO—C(R^{c})₂—C(R^{c})(OC(O)—R^{b})—C(R^{c})₂—OC(O)—R^{d},

wherein:
each R^{c} is independently —H or C₁₋₁₈ alkyl, and
each R^{d} is independently C₄₋₂₂ alkyl.

In some embodiments, the composition excludes monoacylglycerides.

In some embodiments, the composition excludes one or more of glycerol, 1,2-propanediol, 1,2-pentanediol, 1,2-hexandiol, 1,2-octanediol, 1,2,6-trihydroxyhexane, 1,3-propanediol, 1,4-butanediol, 2-butene-1,4-diol, 1,3-butanediol, 3-methyl-1,3-butanediol, 1,3-cyclohexanediol, and 2,3-butanediol.

In some embodiments, the composition excludes one or more of glyceryl monostearate, glyceryl monocaprylate, glyceryl monolaurate, glyceryl monoisostearate, diethyl-D-tartrate, diethyl-L-tartrate, dibutyl-L tartrate, decaglyceryl tristearate, and glyceryl monooleate.

In some embodiments, the composition excludes one or more of triethylene glycol, tetraethylene glycol, triethyleneglycol monomethyl ether, diethyleneglycol monomethyl ether, dipropylene glycol, sorbeth-6, 1,3-dihydroxyacetone dimer, and ethylhexyl glycerin.

In some embodiments, the composition excludes hydrophobic vehicles as described herein.

### Hydrophobic Plasticizer

In some embodiments, the hydrophobic plasticizer may be characterized by an HLB of no greater than about 10. For example, the hydrophobic plasticizer may be characterized by an HLB of no greater than 10, no greater than 9, no greater than 8, no greater than 7, no greater than 6, no greater than 5, no greater than 4, no greater than 3, no greater than 2, no greater than 1, or have a value between any of the preceding values, for example, between about 6 and about 8, between about 3 and about 7, or the like. In some embodiments, the hydrophobic plasticizer may be characterized by an HLB less than 5. In some embodiments, the hydrophobic plasticizer may be characterized by an HLB less than 2.

In some embodiments, the hydrophobic plasticizer may include hydrogen-bonding groups separated by greater than three consecutive atoms. For example, the hydrogen-bonding atoms may be separated by 4, 5, 6, 7, 8, 9, 10, 11, 12, 13, 14, 15, 16, 17, 18, 19, 20, 21, 22, 23, 24, or 25 consecutive atoms, or a number within a range between any of the preceding values, e.g., between about 16 and about 18, between about 4 and about 8, or the like. Without wishing to be bound by theory, hydrogen-bonding atoms are separated by flexible chains of consecutive atoms that adopt chair- or pseudo-chair-like stable configuration. In other words, hydrogen-bonding atoms are separated in such a manner to allow for chelation of the antiseptic compound, e.g., chlorhexidine gluconate and octenidine hydrochloride. For example, hydrogen-bonding groups separated by 4 consecutive atoms may adopt a 7-membered ring conformation, whereas separation of hydrogen-bonding groups by 7 consecutive atoms may adopt a decalin-like chair configuration.

In some embodiments, the hydrophobic plasticizer may be characterized by a hydroxyl value of at least about 20. In some embodiments, the hydrophobic plasticizer may be characterized by a hydroxyl value of about 20 to about 70. In some embodiments, the hydrophobic plasticizer may be characterized by a hydroxyl value of about 20, 25, 30, 35, 40, 45, 50, 55, 60, 65, 70, or a value within a range between any of the preceding values, for example, between about 30 and about 70, between about 40 and about 50, or the like.

In some embodiments, the hydrophobic plasticizer may be characterized by an average molecular weight of about 0.5 kg/mol to about 6 kg/mol. In some embodiments, the hydrophobic plasticizer may have an average molecular weight in kg/mol of about 0.5, 1, 1.5, 2, 2.5, 3, 3.5, 4, 4.5, 5, 5.5 or 6, or a value between any of the preceding values, for example, between about 1 and 5, between about 2 and 4, or the like.

In some embodiments, the hydrophobic plasticizer may solubilize chlorhexidine gluconate, octenidine hydrochloride, or a combination thereof in an amount greater than about 1 wt % with respect to the weight of the hydrophobic plasticizer at a temperature of about 20° C. to about 23° C. For example, the hydrophobic plasticizer may solubilize chlorhexidine gluconate, octenidine hydrochloride, or a combination thereof (wt % with respect to the weight of hydrophobic plasticizer) at a temperature of about 20° C. to about 23° C. in an amount greater than 1, 2, 3, 4, or 5, or a value within a range of any of the preceding values, for example between about 2 and about 3, between about 3 and about 5, or the like. In some embodiments, the hydrophobic plasticizer may solubilize chlorhexidine gluconate, octenidine hydrochloride, or a combination thereof in an amount up to about 5 wt %, up to about 6 wt %, up to about 7 wt % up to about 8 wt %, up to about 9 wt %, or up to about 10 wt %.

In some embodiments, the hydrophobic plasticizer may include a polymer having at least two hydrogen-bonding groups. For example, the hydrophobic plasticizer may a polymer having 2, 3, 4, 5, 6, 7, 8, 9, or 10 hydrogen-bonding groups.

In some embodiments, the hydrophobic plasticizer may include a polymer having at least one hydrogen-bond donor.

In some embodiments, the hydrophobic plasticizer may include a polymer having at least two hydrogen-bond donors.

In some embodiments, the hydrophobic plasticizer may include a polymer having a hydrogen-bond donor selected from —OR, —O—, —C(O)—, —C(O)—O—, —SR, —N(R)(R), —C(O)—N(R)—, —O—C(O)—N(R)—, and —N(R)—C(O)—N(R)—, wherein each R is independently selected from —H and C₁₋₆ alkyl.

In some embodiments, the hydrophobic plasticizer may include a hydrogen-bond donor selected from —OH and —NH(R), wherein R is selected from —H and C₁₋₆ alkyl.

In some embodiments, the hydrophobic plasticizer may include two —OH groups.

In some embodiments, the hydrophobic plasticizer may be selected from a polyester polyol, a polyalkadiene polyol, and a silicone polyol, or a combination thereof. The polyester polyols, polyalkadiene polyols, and silicone polyols of the present disclosure are described in more detail below.

### Polyester Polyol

In some embodiments, the hydrophobic plasticizer may include a polyester polyol. A person of ordinary skill in the art would readily understand that alcohols (R^{a}OH) may be reacted with acids HOC(O)R^{b} or anhydrides RC(O)OC(O)R² under well-known conditions to produce esters (R^{a}OC(O)R^{b}). A polyester refers to a molecule having more than one ester moiety. A polyol refers to a molecule having more than one alcohol moiety. Thus, a polyester polyol refers to a molecule having more than one ester and more than one alcohol.

In some embodiments, the hydrophobic plasticizer may include a polyester polyol having, on average, at least at least two hydroxyl (i.e., —OH) groups. In some embodiments, the polyester polyol may have a hydroxyl value of about 20 to about 70. In some embodiments, the polyester polyol may have a hydroxyl value of about 20, 25, 30, 35, 40, 45, 50, 55, 60, 65, or 70, or a value within a range of any of the preceding values, for example, between about 25 and about 50, between about 20 and about 30, or the like.

In some embodiments, the hydrophobic plasticizer may include a polyester polyol having a molecular weight of about 1500 to about 4000 kg/mol. In some embodiments, the polyester polyol may have a molecular weight in kg/mol of about 1500, 1600, 1700, 1800, 1900, 2000, 2200, 2400, 2600, 2800, 3000, 3200, 3400, 3600, 3800, or 4000, or a value within any of the preceding values for example, between about 2000 and about 3000, between about 1800 and about 3600, or the like.

In some embodiments, the hydrophobic plasticizer may include a polyester polyol represented by Formula (I):

HO-(A-B)ₙ—OH (I),

wherein:
each A is independently selected from C₂-C₂₂ alkylene, C₂-C₂₂ alkenylene, -A′-(cyclohexylenyl)-A′-, -A′-(cyclohexenyl)-A′-, and phenylene,
wherein the C₂-C₂₂ alkylene, the C₂-C₂₂ alkenylene the cyclohexylenyl or the cyclohexenyl are optionally substituted with one or more of C₁-C₂₀ alkyl, and C₂-C₂₀ alkenyl;
each A′ is independently selected from C₂-C₁₆ alkylene and C₂-C₁₆ alkenylene;
each B is independently selected from —C(O)O— and —OC(O)—; and
n is an integer from 4 to 10,
wherein at least one A is selected from a C₁₄-C₂₂ alkylene substituted optionally substituted with one or more of C₁-C₂₀ alkyl and C₂-C₁₆ alkenylene, a C₁₄-C₂₂ alkenylene optionally substituted with one or more of C₁-C₂₀ alkyl and C₂-C₁₆ alkenylene, -A′-(cyclohexylenyl)-A′-, and -A′-(cyclohexenyl)-A′-.

In some embodiments, the polyester polyol may be derived from a C₁₆-C₄₄ alkyl acid, a C₁₆-C₄₄ alkyl diacid, a C₂-C₁₂ alkyl acid, a C₂-C₁₂ alkenyl acid a C₂-C₁₂ alkyl diacid, a C₂-C₁₂ alkenyl diacid, a C₃-C₁₂ alkyl triacid, C₆-C₁₀ aryl acid, a C₆-C₁₀ aryl diacid, a C₆-C₁₀ aryl triacid, C₁₆-C₄₄ alkanol, a C₁₆-C₄₄ alkyl diol, a C₂-C₁₀ alkanol, a C₂-C₁₀ alkyl diol, a C₃-C₁₀ alkyl triol, polyalkylene glycol, or a combination thereof.

In some embodiments, the polyester polyol may be derived from oxalic acid, malonic acid, maleic acid, fumaric acid, succinic acid, glutaric acid, adipic acid, pimelic acid, suberic acid, azelaic acid, sebacic acid, undecanedioic acid, dodecanedioic acid, methanetricarboxylic acid, citric acid, aconitic acid, isocitric acid, tricarballylic acid, 1,3,5-trimethylcyclohexane-1,3,5-tricarboxylic acid, terephthalic acid, phthalic acid, isophthalic acid, ethylene glycol, polyethylene glycol, propylene glycol, polypropylene glycol, 1,4-butane diol, 1,5-pentane diol, 1,6-hexane diol, glycerol, or a combination thereof, or the like.

In some embodiments, the polyester may be derived from C₁₆-C₄₄ alkyl acid, a C₁₆-C₄₄ alkyl diacid, a C₂-C₁₂ alkyl acid, a C₂-C₁₂ alkyl diacid, a C₃-C₁₂ alkyl triacid, C₆-C₁₀ aryl acid, a C₆-C₁₀ aryl diacid, a C₆-C₁₀ aryl triacid, C₁₆-C₄₄ alkanol, a C₁₆-C₄₄ alkyl diol, a C₂-C₁₀ alkanol, a C₂-C₁₀ alkyl diol, a C₃-C₁₀ alkyl triol, oxalic acid, malonic acid, maleic acid, fumaric acid, succinic acid, glutaric acid, adipic acid, pimelic acid, suberic acid, azelaic acid, sebacic acid, undecanedioic acid, dodecanedioic acid, methanetricarboxylic acid, citric acid, aconitic acid, isocitric acid, tricarballylic acid, 1,3,5-trimethylcyclohexane-1,3,5-tricarboxylic acid, terephthalic acid, phthalic acid, isophthalic acid, ethylene glycol, polyethylene glycol, propylene glycol, polypropylene glycol, 1,4-butane diol, 1,5-pentane diol, 1,6-hexane diol, glycerol, or a combination thereof.

In some embodiments, the hydrophobic plasticizer may include a polyester polyol derived from one or more of a C₁₆-C₄₄ alkyl acid and a C₁₆-C₄₄ alkyl diacid. In some embodiments, the hydrophobic plasticizer may include a polyester polyol derived from C₃₆ alkyl diacid and a C₃₆ alkyl diol.

In some embodiments, the hydrophobic plasticizer may include a polyester polyol derived from one or more of a C₂-C₁₂ alkyl acid, a C₂-C₁₂ alkyl diacid, and a C₃-C₁₂ alkyl triacid. In some embodiments, the hydrophobic plasticizer may include a polyester polyol derived from oxalic acid, malonic acid, succinic acid, glutaric acid, adipic acid, pimelic acid, suberic acid, azelaic acid, sebacic acid, undecanedioic acid, dodecanedioic acid, methanetricarboxylic acid, citric acid, aconitic acid, isocitric acid, tricarballylic acid, 1,3,5-trimethylcyclohexane-1,3,5-tricarboxylic acid, or a combination thereof.

In some embodiments, the hydrophobic plasticizer may include a polyester polyol derived from one or more of a C₆-C₁₀ aryl acid, a C₆-C₁₀ aryl diacid, and a C₆-C₁₀ aryl triacid. In some embodiments, the hydrophobic plasticizer may include a polyester polyol derived from terephthalic acid, phthalic acid, isophthalic acid, or a combination thereof.

In some embodiments, the hydrophobic plasticizer may include a polyester polyol derived from one or more of C₁₆-C₄₄ alkanol and a C₁₆-C₄₄ alkyl diol. In some embodiments, the hydrophobic plasticizer may include a polyester polyol derived from or a combination thereof.

In some embodiments, the hydrophobic plasticizer may include a polyester polyol derived from one or more of a C₂-C₁₀ alkanol, a C₂-C₁₀ alkyl diol, and a C₃-C₁₀ alkyl triol. In some embodiments, the hydrophobic plasticizer may include a polyester polyol derived from ethylene glycol, polyethylene glycol, propylene glycol, polypropylene glycol, 1,4-butane diol, 1,5-pentane diol, 1,6-hexane diol, glycerol, or a combination thereof.

In some embodiments, the hydrophobic plasticizer may include a polyester polyol derived from one or more of dimer acid, adipic acid, and 1,6-hexane diol.

In some embodiments, the hydrophobic plasticizer may include a polyester polyol derived from one or more of dimer acid and ethylene glycol.

In some embodiments, the hydrophobic plasticizer may include a polyester polyol derived from one or more of dimer acid, adipic acid, and ethylene glycol.

In some embodiments, the hydrophobic plasticizer may include a polyester polyol derived from one or more of dimer acid and dimer diol.

In some embodiments, the hydrophobic plasticizer may include a polyester polyol derived from one or more of dimer diol and isophthalic acid.

In some embodiments, the hydrophobic plasticizer may include a polyester polyol derived from one or more of dimer diol and terephthalic acid.

In some embodiments, the hydrophobic plasticizer may include or include one or more polyester polyol formulation sold under the tradename PRIPLAST™ (e.g., Priplast 3186, Priplast 3190, Priplast 3192, Priplast 3196, Priplast 3197, and Priplast 3238).

### Polyalkadiene Polyol

In some embodiments, the hydrophobic plasticizer may include a polyalkadiene polyol. A polyalkadiene polyol is a polymer derived from one or more conjugated diene monomers (i.e., homopolymers and copolymers) having between about 1.6 to 2 hydroxyl groups (—OH) on average per molecule. A conjugated diene refers to a moiety wherein two olefins (i.e., —CR═CR—) are separated by a single carbon-carbon bond.

In some embodiments, the conjugated dienes used to prepare polyalkadiene polyols of the present disclosure include 2-substituted or 2,3-disubstituted 1,3-dienes having 4-12 carbon atoms. Example conjugated dienes include butadiene, isoprene, chloroprene, 2-cyano-1,3-butadiene, 2,3-dimethyl-1,2-butadiene, and the like.

In some embodiments, the polyalkadiene polyols are hydroxy-terminated. Hydroxy-terminated polyalkadiene polyols may be prepared according to U.S. Pat. No. 5,416,168 (incorporated herein by reference in its entirety), wherein lithiated silyl-protected alkanols are employed as initiators (and later deprotected), and the polymer chain is terminated with ethylene oxide.

In some embodiments, the polyalkadiene polyols may have about 30-70% 1,4-addition (e.g., I+T+CH₂═CH—CH═CH₂→R—CH₂—CH═CH—CH₂-T). Preference for 1,4-addition (as opposed to 1,2 addition) may be afforded by selection of conditions known in the art. For example, polybutadiene having 30-70% 1,4-addition minimizes the viscosity.

In some embodiments, the polyalkadiene polyols may have no less than 80% 1,4-addition. For example, polyisoprene having no less than 80% 1,4 addition provides a suitable T_{g} and viscosity.

In some embodiments, the polyalkadiene polyols are not hydrogenated.

In some embodiments, the polyalkadiene polyols may be further hydrogenated, i.e., at least a portion of the olefins are reduced with hydrogen and a suitable catalyst. In some embodiments, the polyalkadiene polyols are hydrogenated such that 50-95% of the olefins are reduced. For example, 50%, 55%, 60%, 65%, 70%, 75%, 80%, 85%, 90%, or 95%, or a value within a range between any of the preceding values, e.g., between about 90% and about 95%, between about 65% and about 85%, or the like, of the olefins in the polyalkadiene polyol may be reduced via hydrogenation. In some embodiments, 100% of the olefins in the polyalkadiene may be reduced.

In some embodiments, a hydrogenated polyalkadiene polyol may have about 40-60% 1,2-addition (i.e., I+T+CH₂═CH—CH═CH₂→R—CH₂—CH(CH═CH₂)-T). Preference for 1,2-addition (as opposed to 1,2 addition) may be afforded by selection of conditions known in the art. A hydrogenated polyalkadiene polyol, e.g., hydrogenated polybutadiene, having 40-60% 1,2-addition may prevent waxiness of the polymer.

In some embodiments, the hydrophobic plasticizer may include one or more polyalkadiene polyol represented by the following Formula II:

R¹—(CH₂-A)ₙ-R¹ (II),

wherein:
each R¹ is independently selected from C₁-C₆ alkyl substituted with —OH;
each A is independently selected from —C(R²)═C(R²)—, —CH(R²)CH(R²)—, —C(R²)(C(R²)═CH₂)—, —C(R²)(CH(R²)CH₃)—;
each R² is independently selected from —H, halogen, —CN, phenyl, C₁-C₆ alkyl, and C₁-C₆ alkyl substituted with —OH; and
n is an integer selected from 10-200.

In some embodiments, one R¹ is —(CH₂)₆₀H and one R¹ is —CH₂CH₂OH.

In some embodiments, each A is —C(R²)═C(R²)— and each R² is —H.

In some embodiments, each A is —C(R²)═C(R²)— and one R² is —H and one R² is C₁-C₆ alkyl, e.g., —CH₃.

In some embodiments, the polyalkadiene polyol may be characterized by an average molecular weight of about 500 to bout 20,000. For example, the polyalkadien polyol may be characterized by an average molecular weight of about 500, 600, 700, 800, 900, 1000, 2000, 3000, 4000, 5000, 6000, 7000, 8000, 9000, 10000, 12000, 14000, 16000, 18000, or 20000, or a value within a range between any of the preceding values, for example, between about 12000 and about 18000, between about 1000 and about 10000, or the like.

In some embodiments, the polyalkadiene polyol may be characterized by a hydroxyl value of about 250 to about 10,000. For example, the polyalkadiene polyol may be characterized by a hydroxyl values of about 250, 500, 750, 1000, 2000, 3000, 4000, 5000, 6000, 7000, 8000, 9000, or 10000, or a value within a range between any of the preceding values, for example, between about 500 and about 5000, between about 1000 and about 8000, or the like.

In some embodiments, the hydrophobic plasticizer may include a polyalkadiene polyol selected from Poly-BD R-45HT (ARCO Chemicals; Newtown Square, Pennsylvania), Polyvest HT (Evonik Industries; Essen Germany), Krasol LBH 2000 or LBH 3000 (Cray Valley USA LLC; Exton, Pennsylvania).

### Silicone Polyol

In some embodiments, the hydrophobic plasticizer may include a silicone polyol.

In some embodiments, the hydrophobic plasticizer may include one or more silicone polyol represented by the following Formula (III):

R¹—[Si(R²)(R²)-A-]ₙ-R¹ (III),

wherein:
each R¹ and R² are independently selected from —OH, C₁-C₆ alkyl, or C₁-C₆ alkyl substituted with —OH;
each A is independently selected from —O—, C₂-C₁₀ alkyl, and phenyl; and
n is an integer from 10-200.

In some embodiments, each R¹ is —OH.

In some embodiments, each R¹ is independently selected from C₁-C₆ alkyl substituted with —OH. For example, each R¹ may be —CH₂CH₂OH.

In some embodiments, each R² is selected from C₁-C₆ alkyl. For example, each R² may be —CH₃.

In some embodiments, n is 10, 20, 30, 40, 50, 60, 70, 80, 90, 100, 110, 120, 140, 160, 180, or 200, or a value between any of the preceding values, for example, between about 100 and about 160, between about 50 and about 90, or the like.

In some embodiments, the hydrophobic plasticizer may include a silicone polyol characterized by a hydroxyl value of about 10 to about 200. For example, the silicone polyol may be characterized by a hydroxyl values of about 10, 20, 30, 40, 50, 60, 70, 80, 90, 100, 110, 120, 130, 140, 150, 160, 170, 180, 190 or 200, or a value within a range between any of the preceding values, e.g., between about 50 and about 130, between about 20 and about 70, or the like.

In some embodiments, the hydrophobic plasticizer may include a silicone polyol selected from KF-6000, KF-6001, KF-6002, KF-6003, X-21-5841, KF-9701 (all available from Shin-Etsu Chemical Company), and a combination thereof, or the like.

### Antimicrobial Adhesives

In various embodiments, an antimicrobial adhesive is described. The antimicrobial adhesive may include any composition described herein and a pressure-sensitive adhesive.

In some embodiments, the antimicrobial adhesive may consist essentially of the composition and the pressure-sensitive adhesive.

In some embodiments, the antimicrobial adhesive may be characterized by a glass transition temperature (T_{g}) of about −90° C. to about 10° C. In some embodiments, the antimicrobial adhesive may be characterized by a glass transition temperature (° C.) of about −90, −80, −70, −60, −50, −40, −30, −20, −10, −5, 0, 5 or 10, or a value within a range between any of the preceding values, for example, between about −30 and about −5, between about −70 and about 0, or the like. The T_{g} of the antimicrobial adhesive may be afforded by action of the hydrophobic plasticizer on the pressure-sensitive adhesive (i.e., the plasticizer lowers the glass transition temperature of the pressure-sensitive adhesive).

In some embodiments, the antimicrobial adhesive may further include one or more tackifier, antioxidant, pigment, reinforcing filler, cross-linker, and electrolyte.

### Pressure-Sensitive Adhesive

In some embodiments, the pressure-sensitive adhesive may be characterized by a glass transition temperature (T_{g}) of about −70° C. to about 20° C. In some embodiments, the pressure-sensitive adhesive may be characterized by a T_{g} in ° C. of about −70, −60, −50, −40, −30, −20, −10, −5, 0, 5, 10, or 20, or a value within a range between any of the preceding values, for example, between about −20 and about 5, between about −50 and about −30, or the like.

In some embodiments, the pressure-sensitive adhesive is selected from an acrylic polymer or copolymer. In some embodiments, the acrylic polymer or copolymer may be the reaction product one of monomers selected from an alkyl (meth)acrylate, N-vinyl pyrrolidone, N-vinyl caprolactam, (alkyl-substituted)acrylamide, (alkyl-substituted)methacrylamide, 2-hydroxyethyl (meth)acrylate or and a combination thereof.

### Medical Articles

In various embodiments, a medical article is described. The medical article may include a substrate having a first surface and second surface opposite the first surface, and any antimicrobial adhesive described herein disposed on the first surface.

In some embodiments, the substrate may be a polymeric film. The polymeric film may be woven or nonwoven.

In some embodiments, the medical article may further include a release liner in contact with the antimicrobial adhesive. The release liner may protect the antimicrobial adhesive from coming into contact with foreign matter prior to use. The release liner may further facilitate application of the medical article to a surface.

In some embodiments, the medical article may further include a delivery system disposed on the second surface. The delivery system may be, for example, a paper that is releasably secured to the second surface with an adhesive. The delivery system may provide structural integrity to the medical article in order to facilitate application of the medical article to a surface.

In some embodiments, the medical article may be configured in various shapes, including custom shapes for fitting over contoured surfaces.

In some embodiments, the medical article may be in the form of a sheet or a roll.

In some embodiments, the antimicrobial article is a tape or wrap.

In some embodiments, the medical article is a wound dressing. The medical article may be in the shape of any wound dressing known in the art.

In some embodiments, the medical article is an intravenous dressing.

In some embodiments, the medical article is a surgical drape.

### Methods for Preparing Compositions

In various embodiments, a method for preparing a composition described herein is described. The method may include providing chlorhexidine gluconate, octenidine hydrochloride, or a combination thereof and the hydrophobic plasticizer, and contacting the chlorhexidine gluconate to the hydrophobic plasticizer to form the composition.

In some embodiments, the chlorhexidine gluconate, octenidine hydrochloride, or a combination thereof is provided as a solid. In other embodiments, the chlorhexidine gluconate, octenidine hydrochloride, or a combination thereof is provided as an aqueous solution.

In some embodiments, the hydrophobic plasticizer is provided as solution in a volatile nonpolar organic solvent, e.g., pentane, hexane, heptane, or the like.

In some embodiments, the method further includes heating the mixture formed upon contacting the chlorhexidine gluconate, octenidine hydrochloride, or a combination thereof and hydrophobic plasticizer to a temperature (° C.) of about 30, 40, 50, 60, 70, or 80, or a value within a range of any of the preceding values, for example, between about 40 and about 60, between about 50 and about 70, or the like. In some embodiments, the heating may be conducted under vacuum. The heating or heating under vacuum may remove solvent and water. In some embodiments, water may be removed via decantation, for example, with a separatory funnel.

In some embodiments, the method may further include solvent and/or water removal by means of one or more of heating; vacuum, including roto-evaporating or freeze-pump-thaw techniques, distillation or azeotropic distillation, molecular sieves, or the like.

### Methods for Preparing Antimicrobial Adhesives

In various embodiments, a method for preparing an antimicrobial adhesive described herein is described. The method may include providing a composition described herein and a pressure-sensitive adhesive, and contacting the composition and the pressure-sensitive adhesive to form the antimicrobial adhesive.

In some embodiments, the composition may include an amount of water (w/w) in less than about 1:1 with respect to chlorhexidine gluconate, octenidine hydrochloride, or a combination thereof. In some embodiments, water may be present in the composition in an amount (w/w) with respect to chlorhexidine gluconate, octenidine hydrochloride, or a combination thereof of about less than 1:1, 0.9:1, 0.8:1, 0.7:1, 0.6:1, 0.5:1, 0.4:1, 0.3:1, 0.2:1, 0.1:1, 0.05, 0.01:1, 0.005:1, or 0.001:1, or a value within a range between any of the preceding values, for example, between about 0.4:1 and about 0.05:1, between about 0.1:1 and about 0.01:1, or the like. In some embodiments, the composition may include only residual water, i.e., less than about 0.01 wt % water with respect to the weight of the composition.

In some embodiments, the pressure-sensitive adhesive may be provided as a solvent-based solution. The solvent may be any organic solvent that is miscible with the pressure-sensitive adhesive. For example, the solvent may include ethyl acetate, heptane, toluene, and methyl ethyl ketone, a combination thereof, or the like.

### Methods for Preparing Medical Articles

In various embodiments, a method for preparing a medical article described herein is described. The method may include providing an antimicrobial adhesive described herein and a substrate, and coating the antimicrobial adhesive onto the substrate.

In some embodiments, the substrate may be a polymeric backing material, such as thermoplastic polyurethanes (e.g., as sold by ESTANE®). In other embodiments, the substrate may be a release liner, which can be made from a variety of materials such as paper, poly-coated paper, polyester film, high-density polyethylene film, silicone, or the like.

In some embodiments, the antimicrobial adhesive may be coated at a thickness of about 50 to about 525 microns. For example, the thickness may be (in microns) about 50, 75, 100, 125, 150, 175, 200, 225, 250, 275, 300, 325, 350, 375, 400, 425, 450, 475, 500, or 525, or a value between any of the preceding values, for example, between about 100 and about 200, between about 75 and about 350, or the like.

In some embodiments, coating the antimicrobial adhesive onto the substrate may include hand-spreading a uniform layer of the antimicrobial adhesive onto the substrate. The hand-spreading may be done with a knife-edge coater.

In some embodiments, the method may further include drying the antimicrobial adhesive at an elevated temperature for period to form a dried antimicrobial adhesive. For example, the antimicrobial adhesives may be dried at a temperature from about 65° C. to about 93° C. for a period of about 1 min to about 10 min.

In embodiments wherein the substrate may be a polymeric backing material, the method may further include contacting the dried antimicrobial adhesive to a release liner.

In embodiments where the substrate may be a release liner, the method may further include laminating the dried antimicrobial adhesive to a polymeric backing material. The laminating may be performed using nip rollers at room temperature.

### Methods for Disinfecting a Surface

In various embodiments, a method for disinfecting a surface is described. The method may include providing a medical article described herein, and contacting the medical article to the surface for a period.

In some embodiments, the surface may be skin or tissue. In some embodiments, the skin or tissue is mammalian skin or tissue. In some embodiments, the tissues may be selected from mucosal tissues, chronic wounds, acute wounds, burns, or the like.

In some embodiments, the skin or tissue may be intact, i.e., undamaged.

In some embodiments, the skin or tissue may be wounded or otherwise damaged.

In some embodiments, the skin or tissue may be intact upon contacting and may remain in contact upon subjecting the skin to damage, e.g., cutting, piercing, or the like.

In other embodiments, the surface may be a medical surface, for example, surgical devices (e.g., scalpel, scissors, blades, forceps, drapes, or the like), medical devices (e.g., catheters, stents, artificial joints, dental implants, or the like), floor tiles, countertops, tubs, dishes, gloves, swabs, cloth, sponges, foams, nonwovens, and paper products.

In some embodiments, the medical articles may be effective against various microorganisms types, e.g., Gram positive bacteria, Gram negative bacteria, fungi, protozoa, mycoplasma, yeast viruses, lipid-enveloped viruses, or the like. For example, the antimicrobial adhesives and medical articles made therefrom may be effective at reducing the number of microorganisms present on the surface and/or preventing the growth of such microorganisms, e.g., Staphylococcus spp., Streptococcus spp., Pseudomonas spp., Enterococcus spp., Escherichia spp., Aspergillus spp., Fusarium spp., Candida spp., Staphylococcus aureus, methicillin-resistant Staphylococcus aureus (MRSA), Staphylococcus epidermidis, Streptococcus pneumoniae, Enterococcus faecalis, vancomycin-resistant Enterococcus (VRE), Pseudomonas aeruginosa, Esherichia coli, Aspergillus niger, Aspergillus fumigatus, Aspergillus clavatus, Fusarium solani, Fusarium oxysporum, Fusarium chlamydosporum, Candida albicans, Candid glabrata, Candida krusei, or the like.

In some embodiments, the medical article may contact the surface for a period in minutes of about 30, 60, 90, 120, 150, 180, or 210 or a value between any of the preceding values, for example, between about 30 and about 120, between about 90 and about 180, or the like.

In other embodiments, the antimicrobial article may contact the surface for a period in hours of greater than about 1, 2, 3, 4, 5, 12, or 24, or a value between any of the preceding values, for example between about 2 and about 5, between about 12 and about 24, or the like. In some embodiments, the antimicrobial article may contact the surface for a period in days of about 1, 2, 3, 4, 5, 6, or 7, or a value between any of the preceding values, for example, between about 1 and about 2, between about 2 and about 5, or the like.

In some embodiments, the method may further include applying water to the surface prior to contacting the medical article to the surface. Wetting the surface with water may facilitate CHG or octenidine migration to the surface of the antimicrobial adhesive.

In some embodiments, the method may be effective to deliver chlorhexidine gluconate, octenidine hydrochloride, or a combination thereof to the surface at an average rate of greater than 15 mcg/sq in per hour. For example, the method may be effective to deliver chlorhexidine gluconate, octenidine hydrochloride, or a combination thereof to the surface at an average rate (mcg/sq. in per hour) of about 15, 20, 25, 30, 35, 40, 45, 50, 60, 70, 80, 90, or 100, or a value between any of the preceding values, for example, between about 30 and about 50, between about 20 and about 60, or the like. The amount of chlorhexidine gluconate, octenidine hydrochloride, or a combination thereof delivered to the surface may be determined using the Surface Availability Analysis described herein.

In some embodiments, the method may be a method for preparing a surface for incision, e.g., surgery.

In some embodiments, the method may be a method for preparing a surface for needle penetration, e.g., to administer intravenous pharmaceuticals or fluids, withdraw fluids, or the like.

### Kits

In various embodiments, a kit is described. The kit may include a medical article described herein and a set of instructions directing a user to disinfect a surface according to the methods described herein.

In various embodiments, a kit is described. The kit may include a medical article described herein and a set of instruction directed a user to prepare a surface for surgery according to the methods described herein.

### EXAMPLES

Objects and advantages of this disclosure are further illustrated by the following examples, but the particular materials and amounts thereof recited in these examples, as well as other conditions and details, should not be construed to unduly limit this disclosure. These examples are merely for illustrative purposes only and are not meant to be limiting on the scope of the appended claims.

All parts, percentages, ratios, etc. in the examples and the rest of the specification are by weight, unless noted otherwise. Materials used in the Examples and their sources are provided in Table 1. Solvents and other reagents used were obtained from Sigma-Aldrich Chemical Company, St. Louis, MO, unless otherwise noted. Unless otherwise indicated, all water is distilled water, and all molecular weights are weight average molecular weight.

Materials used in the preparation of the Examples are summarized in Table 1.

TABLE 1
Materials.

| Name | Description | Trade Name and Source |
|---|---|---|
| Priplast 3186 | Amorphous polyester polyol | Croda Inc., Edison, NJ |
| Priplast 3190 | Amorphous polyester polyol | Croda Inc., Edison, NJ |
| Priplast 3196 | Amorphous polyester polyol | Croda Inc., Edison, NJ |
| Priplast 3197 | Amorphous polyester polyol | Croda Inc., Edison, NJ |
| Priplast 3238 | Amorphous polyester polyol | Croda Inc., Edison, NJ |
| Pripol 2033 | C18 dimer diol | Croda Inc., Edison, NJ |
| GMIS | Glycerol monoisostearate | Croda Inc., Edison, NJ |
| PSA | Poly(isooctyl acrylate/N-vinylpyrrollidone) | 3M Company, St. Paul, Minnesota |
| CHG | Chlorhexidine gluconate, 20% w/v solution | Medichem S.A., Barcelona, Spain |
| | in water | |
| MSSA | Methicillin-sensitive Staphylococcus aureus | American Type Culture Collection |
| | | (ATCC # 25923), |
| | | Manassas, Virginia |
| E. Coli | Escherichia coli | American Type Culture Collection |
| | | (ATCC #11229), |
| | | Manassas, Virginia |
| C. Albicans | Candida Albicans | American Type Culture Collection |
| | | (ATCC #10231), |
| | | Manassas, Virginia |
| Estane 58309 | Polyurethane elastomer film | Lubrizol Advanced Materials, Inc. |
| Ioban | Ioban 2 antimicrobial incise drape | 3M Company, St. Paul, Minnesota |
| Octenidine | Octenidine Hydrochloride | Alfa Aesar (USA) |
| Hydrochloride | | |

### Example 1: Solubility Screening

Screening tests were conducted to determine the solubility of chlorhexidine gluconate (CHG) in hydrophobic vehicles. The screening was performed by diluting the hydrophobic vehicle in n-heptane and then suspending an appropriate amount of aqueous 20% w/v CHG in the solution. A vial containing the resulting suspensions was placed on a hotplate equipped with a magnetic stirrer. It was heated at 65° C. under constant stirring to flash off the heptane and water. After removal of the heptane and water, the nonvolatile residue comprising the hydrophobic vehicle with chlorhexidine gluconate was then applied to a glass microscope slide, cooled to room temperature, and observed in a thin section for transparency and clarity. Solubility was determined by visual observation wherein a clear, translucent appearance indicated solubility of CHG in the hydrophobic vehicle. HLB values were estimated from molecular weights and structures, where available. Where structures were not available the molecules were estimated to have a maximum of 4 ester and 2 hydroxyl groups.

TABLE 2
Solubility of CHG and octenidine hydrochloride at various weight
percentages in hydrophobic vehicles and estimated HLB values.

| Hydrophobic | | | | | 10% |
|---|---|---|---|---|---|
| α,ω-diol | Estimated | | | | Octenidine |
| (vehicle) | HLB | 1% CHG | 2% CHG | 4% CHG | HCL |
| Priplast 3197 | 1.2 | Clear, translucent | Clear, translucent | Hazy | |
| Priplast 3196 | 1.4 | Clear, translucent | Clear, translucent | Slight haze | |
| Pripol 2033 | | | | | Clear, |
| | | | | | translucent |

### Adhesive Preparation Procedure

Adhesive compositions were prepared by blending together a solvent-based pressure sensitive adhesive (poly(isooctyl acrylate/N-vinylpyrrolidone) in an ethyl acetate/heptane/methanol blend obtained from 3M Company, St. Paul, MN), an aqueous solution of CHG, and a hydrophobic diol through simple manual agitation.

### Adhesive Coating Procedure

Adhesive compositions were coated as hand-spreads by applying a uniform layer of the prepared adhesive composition on the release surface of a suitable release liner using a knife-edge coater. The wet adhesive thickness ranged from 50 to 510 microns (2-20 mils). The coated adhesive compositions were dried in a solvent oven for 1-10 minutes at temperatures between 65° C. and 93° C. (150° F. and 200° F.).

### Adhesive Lamination Procedure

The dried adhesives were used to prepare adhesive articles by laminating the dried adhesive to 0.8 mil. thick Estane 58309 film using nip rollers at room temperature.

### Surface Availability Analysis

In some embodiments, a discrete amount of CHG should be available at the surface of the adhesive. Surface availability was determined by exposing the surface of the dried adhesive to water in a resting state, according to the following method. A sample of an adhesive article sufficient to cover a circular area of 5.07 square centimeters was cut from a larger section of an adhesive article prepared as described above. The release liner was removed, exposing the surface of the dried adhesive, and water (50 mcL) was pipetted on the adhesive surface and uniformly spread to cover an area of 2.54 square centimeters. The sample was then incubated at 35° C. in a humidified incubator for 90 minutes at about 70% relative humidity. After the desired test time had elapsed, the water was transferred to a liquid chromatography vial for analysis. Samples were analyzed by reverse-phase HPLC using absorbance detection on an Agilent 1200 HPLC system consisting of a quaternary gradient pump, autosampler, heated column compartment and variable wavelength detector. 5.0 mcL portions of sample solutions were injected onto a MACMOD Analytical Inc. 150×3 mm ACE 3 micrometer C18 column. The column was equilibrated with 80/20 v/v water/methanol containing 40 mM pH 3.7 ammonium formate buffer at 0.50 mL/min and 40° C. Following injection, the samples were eluted with a 30 min linear gradient to 20/80 v/v water/methanol containing 40 mM pH 3.7 ammonium formate buffer. This eluent composition was held isocratically for 5 minutes before re-equilibration in the starting eluent. Absorbance detection of the 254+2 nm signal was utilized to quantify sample concentration of chlorhexidine gluconate against standard solutions containing chlorhexidine acetate (“CHA”). A molar fraction of 1.435 was applied to the quantitation to account for the molar ratio of CHG/CHA (898/626).

### Example 2

The following examples demonstrate the release of CHG from drape samples made with adhesive formulations containing different α,ω-diols (A1-A5) and the effect of diol concentration on release (A5, A6). The compositions of the adhesives used in Samples A1-A6, estimated HLB values, and CHG release results are provided in Table 3.

TABLE 3
Adhesive compositions, estimated HLB, and CHG release results.

| | | | | Adhesive | | | |
|---|---|---|---|---|---|---|---|
| Drape | | Molecular | Estimated | composition (% w/w) | | | CHG release |
| Sample | Diol | weight | HLB | CHG | PSA | Diol | (mcg/sq. in.) |
| A1 | Priplast 3186 | 1700 | 2.5 | 2 | 68 | 30 | 18 |
| A2 | Priplast 3190 | 2000 | 2.1 | 2 | 68 | 30 | 17 |
| A3 | Priplast 3196 | 3000 | 1.4 | 2 | 68 | 30 | 27 |
| A4 | Priplast 3238 | 2000 | 2.1 | 2 | 68 | 30 | 18 |
| A5 | Priplast 3197 | 2000 | 1.2 | 2 | 68 | 30 | 18 |
| A6 | Priplast 3197 | 2000 | 1.2 | 2 | 48 | 50 | 27 |

### Direct Time Kill Analysis

Specimens of several coated adhesives were subjected to antimicrobial performance testing according to the following 90-minute time kill study. A suspension of MSSA, E. coli, or C. albicans was prepared at a concentration of 1×10⁸ colony forming units (CFU) per milliliter (mL) in phosphate buffered water (pbw) using a 0.5 McFarland Equivalence Turbidity Standard. Using an Eppendorf pipette, 50 microliters (L) of this suspension was transferred as 15-16 separate droplets to the adhesive surface of a 2.5 cm diameter section of an adhesive film. These inoculated specimens were then incubated at room temperature (23+/−2° C.) for 5-30 minutes. After incubation, the specimens were placed in 20 mL of neutralizing buffer and sonicated for one minute followed by vortexing for two minutes. Portions of the resulting solution were serially diluted with phosphate buffered water. The neat solution and dilutions were each plated to 3M PETRIFILM aerobic count plates (3M Company, St. Paul, MN) and incubated for at least 24 hours. The 3M PETRIFILM plates were then counted using a 3M PETRIFILM plate reader (model 6499, 3M Company).

### Example 3

Example 3 evaluated the antimicrobial activity of drape samples made with adhesive A5 after aging at room temperature for different lengths of time. In the placebo, the CHG was omitted from composition A5 and replaced with additional base PSA. The results are provided in Table 4. Antimicrobial activity greater than 4 logs was demonstrated in all cases against representative gram positive (MSSA), gram negative (E. coli) and yeast (C. albicans) microorganisms.

TABLE 4
Effect of aging on drape samples made with adhesive A5 on microorganism kill.

| | Age | | Log | Log reduction | Average log |
|---|---|---|---|---|---|
| Drape sample | (months) | Microorganism | reduction | (duplicate) | reduction |
| Placebo | N.A. | MSSA | 0.24 | 0.27 | 0.26 |
| Adhesive A5 | 4 | MSSA | 5.18 | 5.48 | 5.33 |
| Adhesive A5 | 5 | E. coli | 5.57 | 5.57 | 5.57 |
| Adhesive A5 | 10 | C. albicans | 4.17 | 4.14 | 4.15 |

### Knee Flexion Model Study

A Knee Flexion Model was used to evaluate the drape adhesion to skin and drape removal performance of the chlorhexidine gluconate (CHG) antimicrobial incise drapes. The Knee Flexion Model was used to simulate surgical conditions.

In the study, the test subject was sitting or lying on a padded examination table. If an excessive amount of hair existed on the test sites, the areas were clipped prior to the initiation of the study to ensure good sample-skin contact. Both legs were prepped with the desired surgical prep, CHLORAPREP HI-LITE ORANGE (Becton-Dickinson, Franklin Lakes, NJ). After at least 3 minutes air drying of the skin preparation, the legs were draped. Each subject had two 3″×10″ drape samples applied to each leg longitudinally over the knee to the left and right side of the midline. An approximate ¼″-½″ gap was left between the two drapes from about 3″ above the kneecap to 3″ below the knee.

Drape samples were worn for approximately 30 minutes. Drape lift was evaluated after a dry flex challenge (flexing the knees 10 times as far as comfortable and extending straight), a wet flex challenge (applying saline-soaked gauze to cover the midline for 5 minutes followed by 10 knee flexes), and after a pulse lavage challenge (applying 200-300 cc saline solution along the midline between the two drapes using low setting pulse lavage). Areas of drape lift were marked and photographed after each challenge. At the end of the pulse lavage lift assessment, the drape samples and excess skin prep were removed. Assessments of skin condition (irritation and stripping), adhesive residue, ease of removal, and subject's assessment of pain level were performed after drape removal. Geometric mean of lifted area (mm2) and frequency of lift (%) were reported for all test subjects of each clinical study.

### Example 4

Adhesive A5 was used to make a representative CHG drape using the procedures described above. A comparative adhesive (C1) was also used to fabricate a comparative drape. Both adhesive formulations are provided in Table 5. The results of knee flexion model studies of the representative CHG drape, the comparative drape, and a commercially available Ioban drape (3M Company) are provided in Table 6. Lift area (geometric mean of the non-zero values) and drape lift frequency (fraction of drape samples exhibiting any amount of lift) after lavage were observed to be statistically no different for A5 and the 3M Ioban incise drape. The C1 drapes were statistically inferior to both these drapes in lift area and lift frequency, underscoring the advantage of using diol plasticizers where the hydrogen bonding groups are separated by larger than 3 carbon atoms on the molecule.

TABLE 5
Drape adhesive compositions

Adhesive composition(% w/w)

| Example | CHG | PSA | Priplast 3197 | GMIS |
|---|---|---|---|---|
| A5 | 2 | 68 | 30 | — |
| C1 | 2 | 68 | 20 | 10 |

TABLE 6
Area of lift per knee model studies.

| | Geometric mean of the | Drape lift |
|---|---|---|
| Drape sample | non-zero values (mm²) | frequency (%) |
| A5 | 270 | 66 |
| C1 | 1200 | 94 |
| Ioban | 490 | 66 |

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A composition comprising:
chlorhexidine gluconate, octenidine hydrochloride, or a combination thereof; and
a hydrophobic plasticizer having at least two hydrogen-bonding groups separated by greater than three consecutive atoms,
wherein the hydrophobic plasticizer is characterized by a hydrophilic-lipophilic balance of no greater than 10 as determined using the HLB Method, and;
wherein the hydrophobic plasticizer is capable of solubilizing chlorhexidine gluconate in an amount of at least 2 g per 100 g at a temperature of 20-23° C.

2. (canceled)

3. The composition of claim 1, wherein chlorhexidine gluconate, octenidine hydrochloride, or a combination thereof is present in an amount of about 0.2 wt % to about 5 wt % with respect to the weight of the composition.

4. (canceled)

5. The composition of claim 1, wherein chlorhexidine gluconate, octenidine hydrochloride, or a combination thereof is present in an amount of about 0.1 wt % to about 20 wt % with respect to the weight of the hydrophobic plasticizer.

6. (canceled)

7. The composition of claim 1, wherein the hydrophobic plasticizer is present in an amount of about 10 wt % to about 50 wt % with respect to the weight of the composition.

8. The composition of claim 1, wherein the hydrophobic plasticizer is characterized by an average molecular weight of about 0.5 kg/mol to about 6 kg/mol and a hydroxyl value of at least 20.

9-15. (canceled)

16. The composition of claim 1, the hydrophobic plasticizer is a compound represented by one of Formulae (I), (II), or (III):
HO-(A-B)ₙ—OH (I),
wherein:
each A is independently selected from C₂-C₂₂ alkylene, C₂-C₂₂ alkenylene, -A′-(cyclohexylenyl)-A′-, -A′-(cyclohexenyl)-A′-, and phenylene, wherein the C₂-C₂₂ alkylene, the C₂-C₂₂ alkenylene the cyclohexylenyl or the cyclohexenyl are optionally substituted with one or more of C₁-C₂₀ alkyl, and C₂-C₂₀ alkenyl;
each A′ is independently selected from C₂-C₁₆ alkylene and C₂-C₁₆ alkenylene;
each B is independently selected from —C(O)O— and —OC(O)—; and
n is an integer from 4 to 10,
wherein at least one A is selected from a C₁₄-C₂₂ alkylene substituted optionally substituted with one or more of C₁-C₂₀ alkyl and C₂-C₁₆ alkenylene, a C₁₄-C₂₂ alkenylene optionally substituted with one or more of C₁-C₂₀ alkyl and C₂-C₁₆ alkenylene, -A′-(cyclohexylenyl)-A′-, and -A′-(cyclohexenyl)-A′-;
R¹—(CH₂-A)ₙ-R¹ (II),
wherein:
each R¹ is independently selected from C₁-C₆ alkyl substituted with —OH:
each A is independently selected from —C(R²)═C(R²)—, —CH(R²)CH(R²)—, —C(R²)(C(R²)═CH₂)—, —C(R²)(CH(R²)CH₃)—;
each R² is independently selected from —H, halogen, —CN, phenyl, C₁-C₆ alkyl, and C₁-C₆ alkyl substituted with —OH; and
n is an integer selected from 10-200;
R¹—[Si(R²)(R²)-A-]ₙ-R¹ (III),
wherein:
each R¹ and R² are independently selected from —OH, C₁-C₆ alkyl, or C₁-C₆ alkyl substituted with —OH;
each A is independently selected from —O—, C₂—C alkyl, and phenyl; and
n is an integer from 10-200.

17-19. (canceled)

20. The composition of claim 1, wherein the hydrophobic plasticizer is derived from oxalic acid, maleic acid, fumaric acid, malonic acid, succinic acid, glutaric acid, adipic acid, pimelic acid, suberic acid, azelaic acid, sebacic acid, undecanedioic acid, dodecanedioic acid, methanetricarboxylic acid, citric acid, aconitic acid, isocitric acid, tricarballylic acid, 1,3,5-trimethylcyclohexane-1,3,5-tricarboxylic acid, terephthalic acid, phthalic acid, isophthalic acid, ethylene glycol, polyethylene glycol, propylene glycol, polypropylene glycol, 1,4-butane diol, 1,5-pentane diol, 1,6-hexane diol, glycerol, or a combination thereof.

21. The composition of claim 1, wherein the hydrophobic plasticizer is derived from:
a C₁₆-C₄₄ alkyl acid, a C₁₆-C₄₄ alkyl diacid, a C₂-C₁₂ alkyl acid, a C₂-C₁₂ alkenyl acid a C₂-C₁₂ alkyl diacid, a C₂-C₁₂ alkenyl diacid, a C₃-C₁₂ alkyl triacid, C₆-C₁₀ aryl acid, a C₆-C₁₀ aryl diacid, a C₆-C₁₀ aryl triacid, C₁₆-C₄₄ alkanol, a C₁₆-C₄₄ alkyl diol, a C₂-C₁₀ alkanol, a C₂-C₁₀ alkyl diol, a C₃-C₁₀ alkyl triol, polyalkylene glycol, or a combination thereof;
one or more of a C₆-C₁₀ aryl acid, a C₆-C₁₀ aryl diacid, and a C₆-C₁₀ aryl triacid;
terephthalic acid, phthalic acid, isophthalic acid, or a combination thereof;
one or more of a C₁₆-C₄₄ alkanol and a C₁₆-C₄₄ alkyl diol;
one or more of a C₂-C₁₀ alkanol, a C₂-C₁₀ alkyl diol, and a C₃-C₁₀ alkyl triol; or
ethylene glycol, polyethylene glycol, propylene glycol, polypropylene glycol, 1,4-butane diol, 1,5-pentane diol, 1,6-hexane diol, glycerol, or a combination thereof.

22-25. (canceled)

26. The composition of claim 1, wherein the hydrophobic plasticizer is selected from:
a polyester polyol comprising one or more of dimer acid, adipic acid, and 1,6-hexane diol;
a polyester polyol comprising one or more of dimer acid and ethylene glycol;
a polyester polyol comprising one or more of dimer acid, adipic acid, and ethylene glycol;
a polyester polyol comprising one or more of dimer acid and dimer diol;
a polyester polyol comprising one or more of dimer diol and isophthalic acid; and
a polyester polyol comprising one or more of dimer diol and terephthalic acid.

27-31. (canceled)

32. The composition of claim 1, wherein the hydrophobic plasticizer is characterized by an HLB of less than 5.

33. (canceled)

34. The composition of claim 1, further comprising water present in amount of less than about 1 wt % with respect to the weight of the composition; or
further comprising water present in an amount of less than 1:1 by weight with respect to the weight of chlorhexidine gluconate, octenidine hydrochloride, or a combination thereof.

35. (canceled)

36. The composition of claim 1, further comprising a hydrophilic vehicle in an amount less than 0.1:1 by weight with respect to chlorhexidine gluconate, octenidine hydrochloride, or a combination thereof, wherein the hydrophilic vehicle is characterized by a hydrophilic-lipophilic balance greater than 10.

37-38. (canceled)

39. The composition of claim 1, one or more of:
excluding a hydrophilic vehicle, wherein the hydrophilic vehicle is characterized by a hydrophilic-lipophilic balance greater than 10;
excluding C₈₋₁₂ alkyl 1,2-diols and C₈₋₁₂ alkyl 1,3-diols:
excluding compounds of the formula:
HO—C(R^{a})₂—C(R^{a})—OC(O)—R^{b},
wherein:
each R^{a} is independently —H or C₁₋₁₈ alkyl, and
R^{b} is C₄₋₂₂ alkyl; or
excluding compounds of the formulae:
HO—C(R^{c})₂—C(R^{c})(OH)—C(R^{c})₂—OC(O)—R^{d},
HO—C(R^{c})₂—C(R^{c})(OC(O)—R^{b})—C(R^{c})—OH, or
HO—C(R^{c})₂—C(R^{c})(OC(O)—R^{b})—C(R^{c})₂—OC(O)—R^{d},
wherein:
each R^{c} is independently —H or C₁₋₁₈ alkyl, and
each R^{d} is independently C₄₋₂₂ alkyl.

40. (canceled)

41. An antimicrobial adhesive comprising:
a composition of claim 1; and
a pressure-sensitive adhesive.

42. (canceled)

43. The antimicrobial adhesive claim 41, the pressure-sensitive adhesive characterized by a glass transition temperature of −90° C. to about 10° C., or is an acrylic polymer or an acrylic copolymer.

44. (canceled)

45. A medical article comprising:
a substrate having a first surface and second surface opposite the first surface; and
an antimicrobial adhesive of claim 41 disposed on the first surface.

46-48. (canceled)

49. A method for preparing a composition of claim 1, the method comprising:
providing chlorhexidine gluconate, octenidine hydrochloride, or a combination thereof and the hydrophobic plasticizer; and
contacting the chlorhexidine gluconate, octenidine hydrochloride, or a combination thereof to the hydrophobic plasticizer to form the composition.

50. A method for preparing an antimicrobial adhesive, the method comprising:
providing the composition of claim 1;
providing a pressure-sensitive adhesive; and
contacting the composition and the pressure-sensitive adhesive to form the antimicrobial adhesive.

51. A method for preparing a medical article, the method comprising:
providing a substrate and the antimicrobial adhesive of claim 41; and
contacting the antimicrobial adhesive to the substrate to provide the medical article.

52. A method of disinfecting a surface, the method comprising:
contacting a medical article of claim 45 to the surface; and
allowing the medical article to contact the surface for a period.

53-56. (canceled)
